(19) United States
(12) Patent Application Publication
(10) Pub. No.: US 2024/0293304 A1
(43) Pub. Date: Sep. 5, 2024

(54) SULFONATE-FUNCTIONALIZED ALKYL POLYGLUCOSIDES AS SULFATE-FREE SURFACTANTS FOR TOOTHPASTE AND MOUTHWASH

(71) Applicant: Colonial Chemical, Inc., South Pittsburg, TN (US)

(72) Inventors: Andrew Minczuk, South Pittsburg, TN (US); Dennis Abbeduto, South Pittsburg, TN (US); Molly McEnery, South Pittsburg, TN (US); Robert N. Comber, South Pittsburg, TN (US)

(21) Appl. No.: 18/571,699

(22) PCT Filed: Jun. 21, 2022
(86) PCT No.: PCT/US22/34403

§ 371 Date: Dec. 18, 2023

Related U.S. Application Data
(60) Provisional application No. 63/212,558, filed on Jun. 18, 2021.

(51) Int. Cl. A61K 8/73; A61K 8/21; A61Q 11/00

(52) U.S. Cl. CPC A61K 8/73; A61K 8/21; A61Q 11/00; A61K 2800/28

(57) ABSTRACT

The invention relates to the use of sulfonate-functionalized alkyl polyglucosides and other functionalized alkyl polyglucosides surfactants in oral care applications such as toothpaste and mouthwash. These surfactants have desired performance attributes while not displaying the potential to cause oral or gingival irritation neither as a 4% solution, nor as a neat product.

### FIELD OF THE INVENTION AND BACKGROUND

Improvement of toothpaste or mouthwash compositions has been the target of oral care research.

US Published Patent Application No. 2020/0261332 relates to stable anhydrous toothpaste concentrate tablets comprising a binding agent, an abrasive agent, a whitening agent, a polishing agent, a sulfate-free surfactant, natural gum, sweetener, and a flavor agent.

U.S. Pat. No. 9,585,818, Enamel protectant and repair toothpaste treatments, is directed to stannous fluoride toothpaste treatments with improved enamel protectant factor (EPF) values and improved enamel repair factor (ERF) values.

US Published Patent Application No. 2020/0121574 is directed to a non-alcohol mouthwash composition exhibiting desirable advantages of being alcohol free, being sodium lauryl sulfate free, being gluten free, and being substantially 100% natural, as well as alleviating Xerostomia (dry mouth) by stimulating salivary flow, combatting enamel erosion by providing a means of remineralization, providing anticariogenic activity by acting as an antimicrobial agent, and/or providing anti-inflammatory activity, among other benefits.

Alkyl polyglycosides have been used for a number of cleaning applications, including toothpastes and mouthwashes. For example, U.S. Pat. No. 5,734,029 is directed to the preparation of improved alkypolyglycoside surfactant mixtures for a variety of end-use applications, including hard surface cleaners, laundry detergents, and personal care products, such as shampoos, soaps, facial cleansers, foam baths, toothpaste, mouthwashes, antiperspirants and the like.

The anionic surfactant most commonly used in commercial toothpastes is sodium lauryl sulphate, but other anionic surfactants have also been used and these include sodium dodecylbenzene sulphonate, sodium lauroyl sarcosinate and sodium coconut monoglyceride sulphonate. Surfactants need to be used at levels that provide optimum cleaning, foaming and taste. Scheller, in U.S. Pat. No. 4,301,141 notes that toothpastes need a high enough content of foaming surfactants to achieve minimal optical cleaning control with brushing. Scehller notes that high concentrations of surfactant or wetting agent adversely affects toothpaste flavoring characteristics. Furthermore, it has been known that toothpastes with larger amounts of surfactant loosen the epithelia intercellular structure of the mouth mucous membrane and that the mechanical abrasive action of the toothbrush enhances the resulting scaling-off action. Many literature references report the occurrence of epithelized lesions caused by toothpaste or mouthwash surfactants.

Therefore, novel, safe, natural, biodegradable surfactants and/or components with safe oral toxicity profiles remain a target for toothpaste and mouthwash research. The present invention relates to toothpaste and mouthwash compositions containing functionalized alkyl polyglucosides, specifically sulfonated-functionalized alkyl polyglucosides. One aspect of the present invention relates to a functionalized polyglycoside derivative that is made by the crosslinking reaction of a polyglucoside, together with a functionalizing agent that contains a sulfonate group. The preferred polymers are cross-linked, having more than one group per molecule. The sulfonate-functionalized alkyl polyglucosides of the present invention are derived entirely from renewable plant resources and are low-irritation, anionic surfactants with excellent foam and oral toxicity properties.

### SUMMARY OF THE INVENTION

One embodiment of the present invention is an oral care composition comprising a functionalized alkyl polyglucoside surfactant with the ingredients to form of a toothpaste, powder, liquid dentifrice, mouthwash, denture cleanser, chewing gum or candy.

In another embodiment of the invention, the composition comprises a functionalized alkyl polyglucoside surfactant, a toothpaste abrasive, an optional fluoride ingredient, and an optional flavorant. Further, the composition can further comprise at least one of an antibacterial agent, detergent, remineralizer, humectant, anti-calculus agent, sweetener, and an anti-sensitivity agent.

In some embodiments, the functionalized alkyl polyglucoside is one or more of the following functionalized alkyl polyglucosides: quaternary functionalized alkyl polyglucoside, poly quaternary functionalized alkyl polyglucosides, sulfonated functionalized alkyl polyglucoside, poly sulfonate functionalized alkyl polyglucoside, phosphate functionalized alkyl polyglucosides, poly phosphate functionalized alkyl polyglucosides, betaine functionalized alkyl polyglucosides, poly betaine functionalized alkyl polyglucosides, sulfosuccinate functionalized alkyl polyglucosides, poly sulfosuccinate functionalized alkyl polyglucosides, and citrate functionalized alkyl polyglucoside.

In one aspect the functionalized alkyl polyglucoside includes the following structures, as a mixture:

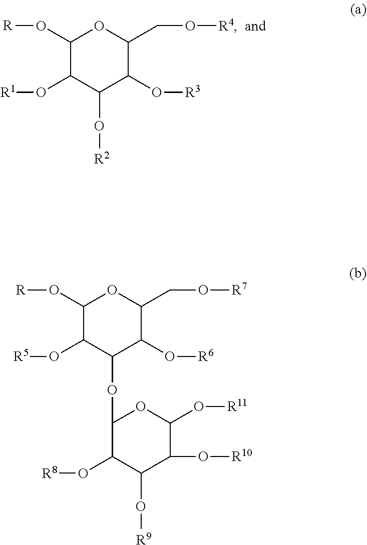

wherein:
R is an alkyl chain having 8 to 22 carbon atoms
R¹, R², R³, R⁴ R⁵, R⁶, R⁷, R⁸, R⁹, R¹⁰, and R¹¹ are independently selected from the group consisting of: —CH₂—C(O)—O⁻M⁺, or —C(O)CH₂—C(O)—O⁻M⁺

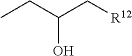

and H, with the proviso that R¹-R¹ are not all H;
R¹² is selected from the group consisting of:
—OH, —SO₃⁻M⁺, and —SO₄⁻²M⁺, —O—P(O)—(OM)₂,
—N(CH₃)₂—R^{1A}, —O—C(O)—CH₂—OH(SO₃⁻M⁺)—C(O)—O⁻M⁺,

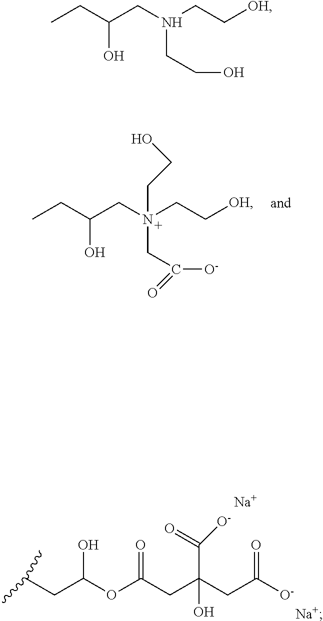

R^{1A} is CH₃—(CH₂)ₙ—;
M is a charge balancing group selected from H, Na, K, or NH₄⁺; and
n is an integer from 0-36;
and positional isomers thereof.

In another aspect, the functionalized alkyl polyglucoside is a cross-linked functionalized alkyl polyglucoside. In one example, the cross-linked functionalized alkyl polyglucoside has the following structures, as a mixture:

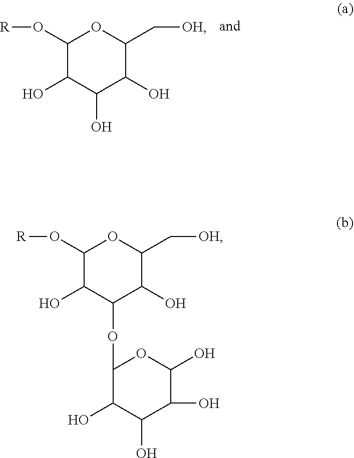

wherein:
R is an alkyl chain having 8 to 22 carbon atoms;
   a crosslinking agent; and
   a functionalizing agent selected from:

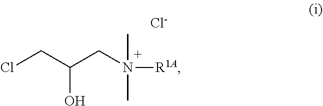

Cl—CH₂—CH(OH)—SO₃M, Cl—CH₂—CH(OH)—SO₄M, Cl—CH₂—CH(OH)—CH₂—OP(O)—(OM)₂, and combinations thereof, wherein R^{1A} is CH₃(CH₂)ₙ—;
(ii) —Cl—CH₂—C(O)⁻Na⁺, 2-halocarboxylic acid, α, β-unsaturated carboxylic acid, cyclic carboxylic acid anhydride, and combinations thereof;

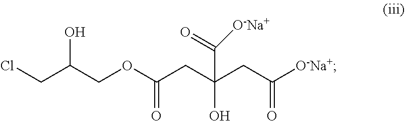

M is a charge balancing group selected from H, Na, K, or NH₄⁺; and
   n is an integer from 0-36; and positional isomers thereof.

The crosslinking agent may be chosen from epichlorohydrin, or Cl—CH₂—CH(OH)—CH₂—Cl.

In another embodiment, the functionalized alkyl polyglucoside is a sulfonate-functionalized alkyl polyglucoside. An example of a sulfonate-functionalized alkyl polyglucoside is a compound of the following formula:

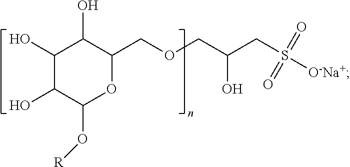

wherein:
R is an alkyl chain;
n is between 1 to about 3, and particularly 1.5;
and positional isomers thereof.

In another embodiment, the sulfonate-functionalized alkyl polyglucoside is chosen from sodium laurylglucoside hydroxypropyl sulfonate, sodium declyglucosides hydroxypropyl sulfonate, and combinations thereof.

In another embodiment of the invention, the functionalized alkyl polyglucoside is a polysulfonate-functionalized alkyl polyglucoside. One example is a sodium hydroxypropylsulfonate laurylglucoside crosspolymer.

In one aspect, the polysulfonate-functionalized alkyl polyglucoside is of the following formula:

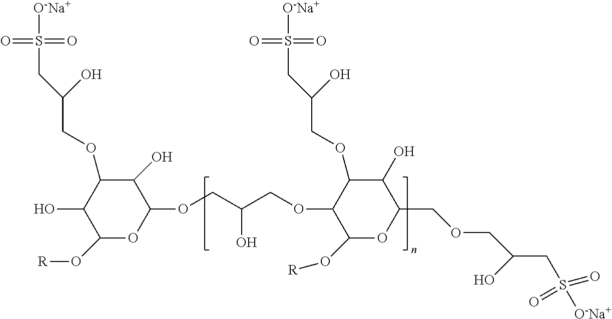

wherein R is an alkyl group having from about 8 to about 22 carbon atoms and n is an integer ranging from 4 to 6; and positional isomers thereof.

In another aspect, the functionalized alkyl polyglucoside is a cross polymer of with an sorbitan ester. One example is a sorbitan oleate decylglucoside crosspolymer.

Here, the cross polymer is represented as compounds of the following formulae, and positional isomers thereof, as a mixture:

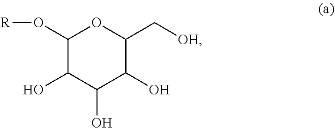

wherein;
R is alkyl having 8 to 22 carbon atoms; and

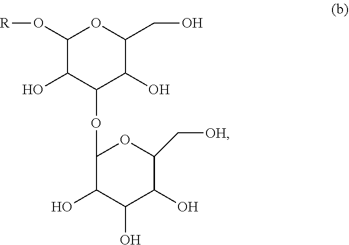

wherein:
R is alkyl having 8 to 22 carbon atoms; and
(c) a sorbitan ester of the following structure:

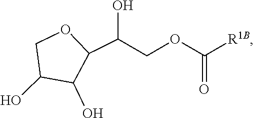

wherein:
R^{1B} is alkyl having 7 to 21 carbons;
   a crosslinking agent of the following structure:

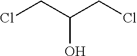

in water; and positional isomers thereof.

The compositions of the invention may further include may known oral care ingredients. For example, the composition may include at least one of abrasives, binders, flavoring agents, coloring agents, humectants, surfactants, fluoride ion sources, anti-calculus agents, alcohol and sweeteners.

### DESCRIPTION OF THE INVENTION

In one embodiment of the present invention, the derivatized polyglucoside of the present invention comprises a monosaccharide unit, a disaccharide unit, a linker, and a functionalizing agent.

At least one functionalized alkyl polyglucoside may be used. Thus, in one embodiment, two, three, four, or more functional alkyl polyglucosides may be blended to comprise the functionalized alkyl polyglucoside component of the cleaning composition of the present invention.

In one example, a derivatized alkyl polyglucoside composition of the present invention includes the following, as a mixture:

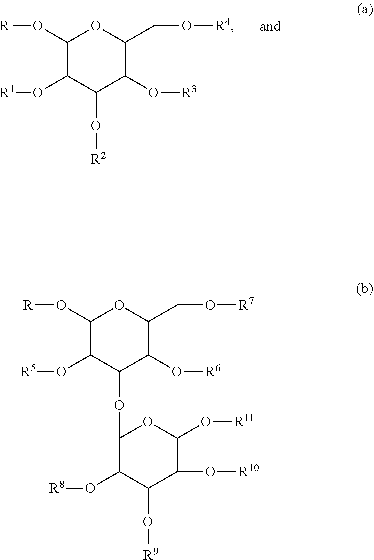

wherein:
R is an alkyl chain having 8 to 22 carbon atoms
R¹, R², R³, R⁴ R⁵, R⁶, R⁷, R¹, R⁹, R¹⁰, and R¹¹ are independently selected from the group consisting of: —CH₂—C(O)—O⁻M⁺, or —C(O)CH₂—C(O)—O⁻M⁺

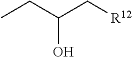

and H, with the proviso that R¹-R¹¹ are not all H;
R¹² is selected from the group consisting of:
—OH, —SO₃⁻M⁺, and —SO₄⁻²M⁺, —O—P(O)—(OM)₂,
—N(CH₃)₂—R^{1A}, —O—C(O)—CH₂—OH(SO₃⁻M⁺)—C(O)—O⁻M⁺,

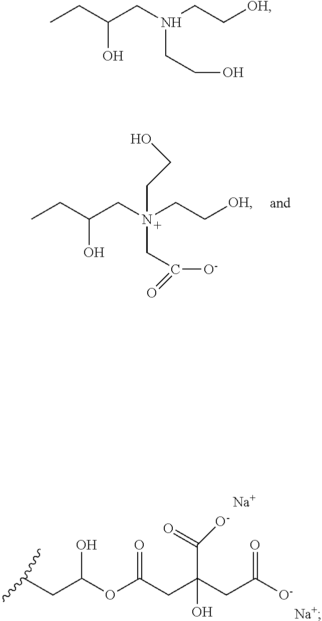

R^{1A} is CH₃—(CH₂)ₙ—;
M is a charge balancing group selected from H, Na, K, or NH₄⁺; and
n is an integer from 0-36;
and positional isomers thereof.

In one embodiment of the present invention, the derivatized polyglucoside of the present invention comprises a monosaccharide unit, a disaccharide unit, a crosslinking agent, and a functionalizing agent.

In this regard, a derivatized alkyl polyglucoside composition of the present invention includes the following, as a mixture:

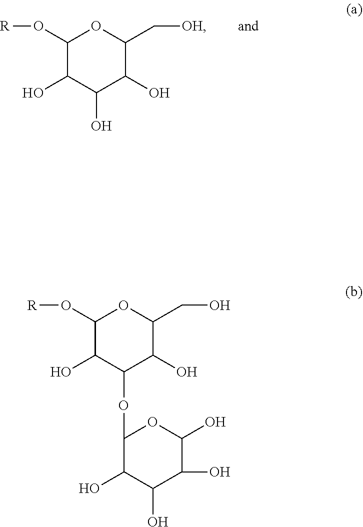

wherein:
R is an alkyl chain having 8 to 22 carbon atoms;
a crosslinking agent; and
a functionalizing agent selected from:

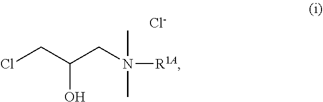

Cl—CH₂—CH(OH)—SO₃M, Cl—CH₂—CH(OH)—SO₄M, Cl—CH₂—CH(OH)—CH₂—OP(O)—(OM)₂, and combinations thereof, wherein
R^{1A} is CH₃(CH₂)ₙ—; (ii) —Cl—CH₂—C(O)⁻Na⁺, 2-halocarboxylic acid, α, β-unsaturated carboxylic acid, cyclic carboxylic acid anhydride, and combinations thereof;

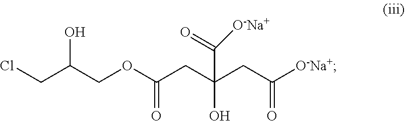

M is a charge balancing group selected from H, Na, K, or NH₄⁺; and
n is an integer from 0-36;
and positional isomers thereof.

In one embodiment of the present invention, the inventive toothpaste or mouthwash surfactant is one that is disclosed in U.S. Pat. No. 6,627,612, incorporated herein by reference and sold by Colonial Chemical, Inc. under the brand name Suga®Nate 160NC, described as sodium laurylglucosides hydroxypropylsulfonate, a 100% biobased surfactant.

In another embodiment of the present invention, the inventive toothpaste or mouthwash surfactant is one that is disclosed in U.S. Pat. No. 7,507,399, incorporated herein by reference and sold by Colonial Chemical, Inc. under the brand name Poly Suga®Nate 160P NC, described as sodium hydroxypropylsulfonate laurylglucoside crosspolymer.

In another embodiment of the present invention, the inventive toothpaste or mouthwash surfactants are those disclosed in U.S. Pat. No. 8,268,766, incorporated herein by reference and sold by Colonial Chemical, Inc. under the brand names Poly Suga®Mulse D6 and Poly Suga®Mulse D9, both described as sorbitan oleate decylglucoside crosspolymer.

In another embodiment of the present invention, the inventive toothpaste or mouthwash surfactant is one that is disclosed in U.S. Pat. No. 6,762,289, incorporated herein by reference and sold by Colonial Chemical, Inc. under the brand name Poly Suga®Sil, described as PEG-8 PG-Coco Glucoside Dimethicone.

In another embodiment of the present invention, the inventive toothpaste or mouthwash surfactant is disclosed in PCT application PCT/US20/62184 describing Citrate-Functionalized Polymeric Surfactants Based Upon Alkyl Polyglucosides, incorporated herein by reference and sold by Colonial Chemical, Inc. under the brand name SugaCitrate L1C, described as Disodium Laurylglucosides Hydroxypropyl Citrate.

Suga®Nate 160NC, described as sodium laurylglucosides hydroxypropylsulfonate, is disclosed in U.S. Pat. No. 6,627,612 and is one of a class of sulfonate-functionalized alkyl polyglucosides whose general structure is shown below.

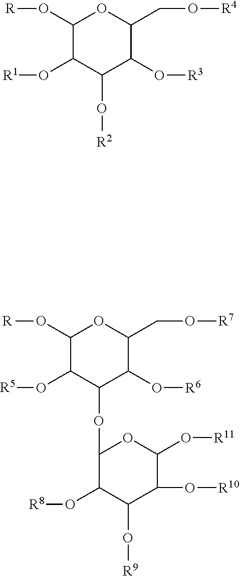

wherein:
   R is an alkyl chain having 8 to 22 carbon atoms;
   R¹, R², R³, R⁴ R⁵, R⁶, R⁷, R⁸, R⁹, R^{1D}, and R¹¹ are independently selected from the group consisting of:

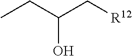

and H, with the proviso that R¹-R¹¹ are not all H;
   R¹² is selected from the group consisting of:
—SO₃⁻M⁺, and —SO₄O⁻²M⁺
M is a charge balancing group selected from H, Na, K, or NH₄⁺

Suga®Nate 160NC is sold as a 40% solution in water but is also sold as Suga®Nate 160 (87% Biobased, 40% solution in water), and Suga®Nate 160 Dry (100% Biobased Surfactant in Anhydrous Form).

A number of functionalized APG's are described in the following patents: sulfonate derivatized alkyl polyglucosides (U.S. Pat. No. 6,627,612, surfactants sold by Colonial Chemical, Inc. under the brand names Suga®Nate), phosphate derivatized alkyl polyglucosides (U.S. Pat. No. 6,627,612, surfactants sold by Colonial Chemical, Inc. under the brand names Suga®Fax) amphoteric glycinate derivatized alkyl polyglucosides (U.S. Pat. No. 6,958,315 surfactants sold by Colonial Chemical under the brand name Suga®Glycinate), and sulfosuccinate derivatized alkyl polyglucosides (U.S. Pat. No. 7,87,571, surfactants sold by Colonial Chemical, Inc. under the brand name Suga®Mates),

Other examples of derivatized alkyl polyglucosides include polysulfonate derivatized alkyl polyglucosides (U.S. Pat. No. 7,507,399, surfactants sold by Colonial Chemical, Inc. under the brand names Poly Suga®Nates), polyphosphate derivatized alkyl polyglucoside (U.S. Pat. No. 7,507,399, surfactants sold by Colonial Chemical, Inc. under the brand names Poly Suga®Phos) polyquaternary derivatized alkyl polyglucosides (U.S. Pat. No. 7,507,399, surfactants sold by Colonial Chemical, Inc. under the brand names Poly Suga®Quats), polycarboxylated derivatized alkyl polyglucosides (U.S. Pat. No. 7,335,627, surfactants sold by Colonial Chemical, Inc. under the brand name Poly Suga®Carb), and polynonionic derivatized alkyl polyglucosides (U.S. Pat. No. 8,268,766, surfactants sold by Colonial Chemical, Inc. under the brand names PolySuga®Mulse).

These functionalized alkyl polyglucosides, or blends thereof, have found wide use in personal care and Household Industrial & Institutional cleaning products. PCT Application PCT/US20/27552, Sugar-Based, Environmentally-Friendly Surfactants for Emulsion Polymerization, describes the use of mild, sulfate-free, ethylene oxide-free functionalized alkyl polyglucosides as emulsion polymerization surfactants. PCT Application PCT/US20/40694, Nonionic Functionalized Poly Alkyl Glucosides as Enhancers For Food Soil Removal, describes the use of blends of functionalized alkyl polyglucosides as effect surface cleaning agents. PCT Application PCT/US20/53150, Blends of Functionalized Poly Alkyl Glucosides as Enhancers for Laundry Soil Removal, describes the use of blends of functionalized alkyl polyglucosides in laundry and spot removal applications. PCT Application PCT/US20/62184, Citrate-Functionalized Polymeric Surfactants Based Upon Alkyl Polyglucosides, describes a new class of functionalized alkyl polyglucosides that can be used in the lot of these cleansing applications.

Traditional toothpaste compositions are well-known in the art. For example, thickeners can be used to give structure and stability to the toothpaste and have a major effect on toothpaste consistency during application to the toothbrush and afterwards during use. Thickeners are often used in combination to give an acceptable consistency, with gums such as xanthan or carrageenan being used together with thickening silicas to achieve the desired results.

Humectants act primarily to help water retention within the toothpaste formulation over time and, as with thickeners, also affect longer-term toothpaste stability and toothpaste consistency during use. Water is one of the main ingredients of most toothpastes and is important for effective fluoride delivery. In addition to this, good water retention will usually give gloss or shine to the formulation and give a more appealing texture to the toothpaste within the mouth. Humectants such as glycerol, sorbitol and polyethylene glycol are typically used to achieve this; all of these have been used for many years in toothpaste formulations for adults and children. These ingredients often also serve other functions in the toothpaste, as most convey some level of sweetness, which is important in compatibility with the flavors for a children's toothpaste.

Surfactants provide foam during brushing, which helps with wetting of the tooth surfaces, dispersion of the toothpaste in the mouth and with loosening of debris and plaque from tissues of the mouth. Choice of surfactant type and concentration for toothpaste is important so as to minimize any potential for irritation of oral soft tissue, and to minimize any negative affect on fluoride availability. Additional surfactants can be added to the compositions of the present invention.

Much of the physical cleaning action during tooth brushing, including removal of plaque and stained pellicle, comes from the use of the toothbrush in combination with abrasive particles in the toothpaste formulation. This cleaning action can vary greatly between different toothpaste formulations and improvements in stain removal, shine and polish can be achieved to a greater or lesser degree depending upon the type, morphology and particle size distribution of the abrasive used. Commonly used abrasive ingredients in toothpaste are various grades of calcium carbonate, silica and alumina.

Flavor is used in toothpaste formulations to make the taste and odor of the product appealing during and after use. Different flavor components can give breath-freshening benefits by masking odors and can provide cooling or warming sensations within the mouth during and after brushing. Flavors can also serve to mask the taste of other ingredients in the toothpaste formulation. Sweeteners, such as sodium saccharin, sucralose and xylitol, work together with the flavor system by adding a degree of sweetness to the toothpaste but avoid the use of fermentable sugars. Xylitol likely has anti-caries potential when delivered continuously from chewing gum and confectionary, although when delivered from toothpaste, evidence for this is inconclusive at present.

Various fluoride salts are used in toothpaste such as sodium fluoride, sodium monofluorophosphate, amine fluoride and stannous fluoride. The main oral health benefit of brushing with fluoride toothpastes is the delivery of fluoride to the mouth.

Other acceptable ingredients that would be used in toothpastes include plaque buffers, such as urea, calcium lactate, calcium glycerophosphate and strontium polyacrylates; anti-inflammatory agents, such as ibuprofen, flurbiprofen, aspirin, and indomethacin; desensitizing agents, such as potassium citrate, potassium chloride, potassium tartrate, potassium bicarbonate, potassium oxalate, potassium nitrate and strontium salts; anti-calculus agents, such as alkali-metal pyrophosphates, hypophosphite-containing polymers, organic phosphonates and phosphocitrates; bio-molecules, such as bacteriocins, antibodies, and enzymes; proteinaceous materials, such as collagen; preservatives; opacifying agents; coloring agents; pH-adjusting agents; sweetening agents, such as saccharin and sorbitol (vide supra); bleaching agents, such as peroxy compounds (e.g. potassium peroxydiphosphate); buffers and salts to buffer the pH and ionic strength of the composition; and pharmaceutically acceptable carriers, e.g. starch, sucrose, water or water/alcohol systems. Water is a particularly preferred pharmaceutically acceptable carrier and is typically present in compositions of the invention at from 5 to 95%, in particular 15 to 75%, and especially at from 35 to 60% by weight of the total composition.

Traditional mouthwash formulations are also well-known in the art. For example, mouthwashes (mouthrinses) are generally classified as either cosmetic or therapeutic or a combination of the two. Cosmetic rinses are commercial products that remove oral debris before or after brushing, temporary suppress bad breath, diminish bacteria in the mouth and refresh the mouth with a pleasant taste. Therapeutic rinses often have the benefits of their cosmetic counterparts, but also contain an added active ingredient, (for example fluoride or chlorhexidine), that help protect against some oral diseases. The amount of the different ingredients in mouthwashes varies from product to product. Some practically have the same composition as toothpastes, although they do not contain abrasives. Distinct from toothpastes most mouth-washes contain alcohol, as a preservative and a semi-active ingredient. The amount of alcohol is usually ranging from 18-26%.

See Vranic et al., Bosn J Basic Med Sci 2004 November; 4(4): 51-58 for additional information concerning possible ingredients for the compositions of the present invention.

### Examples

We now report the use of several of the above functionalized alkyl polyglucosides in toothpaste and mouthwash applications.

### Testing Results: Oral and Gingival Irritation Potential

Suga®Nate 160NC has been tested for its oral and gingival irritation potential in vitro (MatTek EpiOral™ & EpiGingival™). The results indicate that Suga®Nate 160NC does not have the potential to cause oral or gingival irritation neither as a 4% solution nor as a neat product (no ET-50 can be calculated). 2% SLS, a commonly used surfactant in toothpaste formulations, resulted in gingival irritation (ET-50 of 418.2 minutes). Taken together, the results reveal that Suga®Nate 160NC, even as a neat product, is less irritating to oral and gingival tissues than 2% SLS. This data has been integrated into a wider safety assessment which concluded that Suga®Nate 160NC has a safety profile that is favorable to its use in oral care products.

### Toothpaste and Mouthwash Formulations

Example toothpaste and mouthwash formulations using Suga®Nate 160NC were prepared and are shown below in Tables 1 and 2.

TABLE 1
Toothpaste containing Suga ®Nate 160NC

| Ingredients | Wt. % |
|---|---|
| VEEGUM ® D Magnesium Aluminum Silicate | 1.00 |
| Water | 14.20 |
| Sorbitol, 70% | 25.00 |
| Glycerin, USP | 10.00 |
| Sodium Carboxymethyl Cellulose | 0.40 |
| (Aqualon ® CMC 7MF1) | |
| Calcium Carbonate | 44.00 |
| Flavor | 1.00 |
| Sodium Saccharin | 0.20 |
| Sodium Benzoate | 0.20 |
| Suga ®Nate 160NC | 4.00 |

The use of the above toothpaste did not produce any bad taste during or after use.

TABLE 2
Mouthwash containing Suga ®Nate 160NC

| Ingredient | Wt. % |
|---|---|
| Suga ®Nate 160NC | 0.20 |
| Flavor | 0.20 |
| Water | 85.41 |
| Alcohol | 10.00 |
| Sorbitol 70% | 4.00 |
| Sodium Saccharin | 0.07 |
| Benzoic Acid | 0.12 |
| Dyes | qs |

The use of the above mouthwash did not produce any bad taste either during or after use.

Other acceptable ingredients that could be used in mouthwashes include a sweetener, a flavoring agent, a coloring agent, a dispersing agent, a thickening agent, enzymes, an anionic surfactant, a nonionic surfactant, an anti-plaque ingredient, an antimicrobial agent, a calcium ion source, a phosphate ion source, a fluoride source, anti-sensitive teeth agents, water-soluble organic alcohols or glycols, and water.

Eye and skin irritation data available for related functionalized alkyl polyglucosides in this application suggest they may also be safe for inclusion in toothpaste and mouthwash formulations.

Poly Suga®Nate 160P NC, described as sodium hydroxypropylsulfonate laurylglucoside crosspolymer, has a similar eye and skin safety profile as Suga®Nate 160NC. Eye Irritation:

MatTek Epi-Ocular: In vitro epidermal keratinocytes: Results indicate ‘non-irritating’ classification (ET-50: 256 min+).

Poly Suga®Mulse D9, described as sorbitan oleate decylglucoside crosspolymer, has a similar safety profile to other functionalized alkyl polyglucosides described herein. Eye Irritation:

MatTek EpiOcular™: In vitro epidermal keratinocytes: Results indicate a ‘non-irritating’ classification (ET-50: 256 min+).

HET-CAM—Hen's Egg Chorioallantoic Membrane Test, practically no ocular irritation, rated 2.25.

Acute Skin Irritation 48 and 72 Hour Occlusive skin patch test on human volunteers, 53 Test Subjects. 53 out of 53 showed no visible skin reaction (0). No potential for dermal irritation.

Similar eye and skin irritation data is available for Poly Suga®Sil, described as PEG-8 PG-Coco Glucoside Dimethicone.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers. As stated above, the compounds described herein specifically include positional isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

The invention thus being described, it will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only.

We claim:

1. An oral care composition comprising a functionalized alkyl polyglucoside surfactant with the ingredients to form of a toothpaste, powder, liquid dentifrice, mouthwash, denture cleanser, chewing gum or candy.

2. The composition of claim 1, comprising a functionalized alkyl polyglucoside surfactant, a toothpaste abrasive, an optional fluoride ingredient, and an optional flavorant.

3. The composition of claim 2, wherein the composition further comprises at least one of an antibacterial agent, detergent, remineralizer, humectant, anti-calculus agent, sweetener, and an anti-sensitivity agent.

4. The composition of claim 1, wherein the functionalized alkyl polyglucoside is one or more of the following functionalized alkyl polyglucosides: quaternary functionalized alkyl polyglucoside, poly quaternary functionalized alkyl polyglucosides, sulfonated functionalized alkyl polyglucoside, poly sulfonate functionalized alkyl polyglucoside, phosphate functionalized alkyl polyglucosides, poly phosphate functionalized alkyl polyglucosides, betaine functionalized alkyl polyglucosides, poly betaine functionalized alkyl polyglucosides, sulfosuccinate functionalized alkyl polyglucosides, poly sulfosuccinate functionalized alkyl polyglucosides, and citrate functionalized alkyl polyglucoside.

5. The composition of claim 1, the functionalized alkyl polyglucoside being of the following structures, as a mixture: 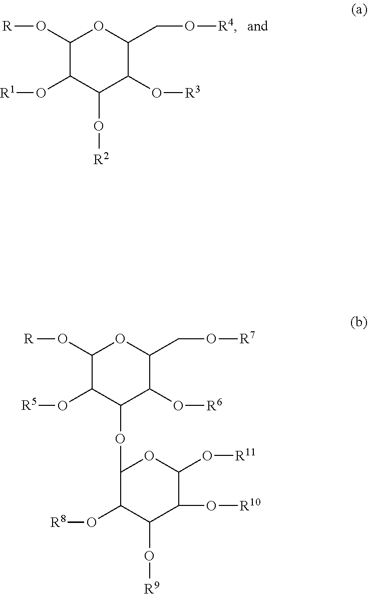
wherein:
R is an alkyl chain having 8 to 22 carbon atoms
R¹, R², R³, R⁴ R⁵, R⁶, R⁷, R¹, R⁹, R¹⁰, and R¹¹ are independently selected from the group consisting of: —CH₂—C(O)—O⁻M⁺, or —C(O)CH₂—C(O)—O⁻M⁺ 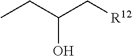
and H, with the proviso that R¹-R¹¹ are not all H;
R¹² is selected from the group consisting of:
—OH, —SO₃M⁺, and —SO₄⁻²M⁺, —O—P(O)—(OM)₂,
—N(CH₃)₂—R^{1A}, —O—C(O)—CH₂—OH(SO₃⁻M⁺)—C(O)—O⁻M⁺, 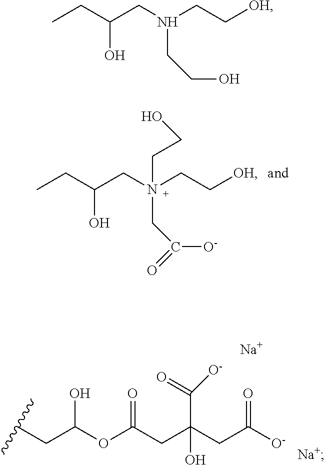
R^{1A} is CH₃—(CH₂)ₙ—;
M is a charge balancing group selected from H, Na, K, or NH₄⁺; and
n is an integer from 0-36;
and positional isomers thereof.

6. The composition of claim 1, wherein the functionalized alkyl polyglucoside is a cross-linked functionalized alkyl polyglucoside.

7. The composition of claim 6, wherein the cross-linked functionalized alkyl polyglucoside has the following structures, as a mixture: 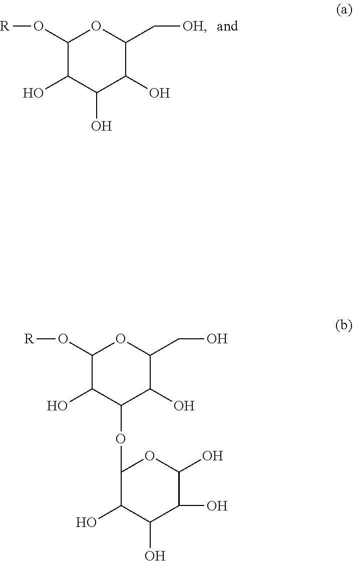
wherein:
R is an alkyl chain having 8 to 22 carbon atoms;
a crosslinking agent; and
a functionalizing agent selected from: 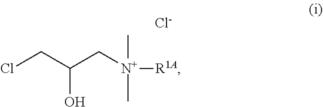
Cl—CH₂—CH(OH)—SO₃M, Cl—CH₂—CH(OH)—SO₄M, Cl—CH₂—CH(OH)—CH₂—OP(O)—(OM)₂, and combinations thereof, wherein
(ii) —Cl—CH₂—C(O)⁻Na⁺, 2-halocarboxylic acid, α, β-unsaturated carboxylic acid, cyclic carboxylic acid anhydride, and combinations thereof; 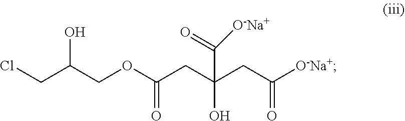
M is a charge balancing group selected from H, Na, K, or NH₄⁺; and
n is an integer from 0-36;
and positional isomers thereof.

8. The composition of claim 7, wherein the crosslinking agent is chosen from epichlorohydrin, or Cl—CH₂—CH(OH)—CH₂—Cl.

9. The composition of claim 1, wherein the functionalized alkyl polyglucoside is a sulfonate-functionalized alkyl polyglucoside.

10. The composition of claim 9, wherein the sulfonate-functionalized alkyl polyglucoside is of the following formula: 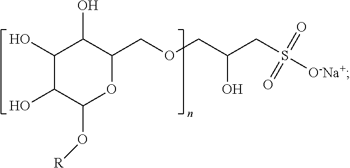
wherein:
R is an alkyl chain;
n is between 1 to about 3, and particularly 1.5;
and positional isomers thereof.

11. The composition of claim 9, wherein the sulfonate-functionalized alkyl polyglucoside is chosen from sodium laurylglucoside hydroxypropyl sulfonate, sodium declyglucosides hydroxypropyl sulfonate, and combinations thereof.

12. The composition of claim 1, wherein the functionalized alkyl polyglucoside is sulfate-free, as well as ethylene oxide and 1,4-dioxane free.

13. The composition of claim 1, wherein the functionalized alkyl polyglucoside is a polysulfonate-functionalized alkyl polyglucoside.

14. The composition of claim 13, wherein polysulfonate-functionalized alkyl polyglucoside is a sodium hydroxypropylsulfonate laurylglucoside crosspolymer.

15. The composition of claim 13, wherein the polysulfonate-functionalized alkyl polyglucoside is of the following formula: 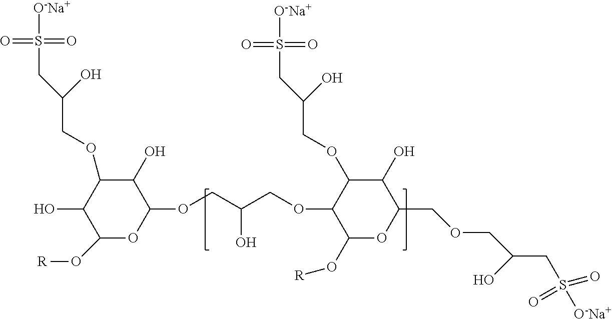
wherein R is an alkyl group having from about 8 to about 22 carbon atoms and n is an integer ranging from 4 to 6; and positional isomers thereof.

16. The composition of claim 1, wherein the functionalized alkyl polyglucoside is a cross polymer of with an sorbitan ester.

17. The composition of claim 16, wherein the functionalized alkyl polyglucoside is a sorbitan oleate decylglucoside crosspolymer.

18. The composition of claim 16, wherein the cross polymer is represented as compounds of the following formulae, and positional isomers thereof, as a mixture: 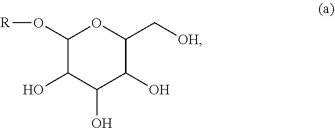
wherein;
R is alkyl having 8 to 22 carbon atoms; and 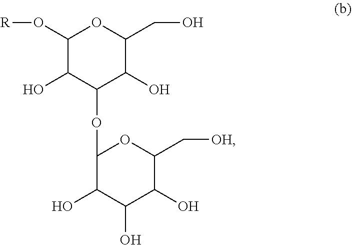
wherein:
R is alkyl having 8 to 22 carbon atoms; and
(c) a sorbitan ester of the following structure: 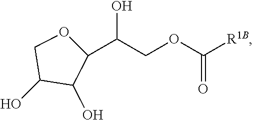
wherein:
R^{1B} is alkyl having 7 to 21 carbons;
a crosslinking agent of the following structure: 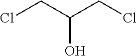
in water; and positional isomers thereof.

19. The composition of claim 1, further comprising at least one of abrasives, binders, flavoring agents, coloring agents, humectants, surfactants, fluoride ion sources, anti-calculus agents and sweeteners.

20. The composition of claim 1, further comprising at least one of alcohol, fluoride, flavoring agent.
